(19) United States
(12) Patent Application Publication
(10) Pub. No.: US 2025/0230133 A1
(43) Pub. Date: Jul. 17, 2025

(54) Luminescent Benzoyl-Pyrazine Organic Compounds

(71) Applicant: The Regents of the University of California, Oakland, CA (US)

(72) Inventors: Ryan D. Baxter, Merced, CA (US); Ryan P. Brisbin, Merced, CA (US); Sayantani Ghosh, Merced, CA (US)

(21) Appl. No.: 18/862,112

(22) PCT Filed: May 1, 2023
(86) PCT No.: PCT/US2023/020583

§ 371 Date: Oct. 31, 2024

Related U.S. Application Data
(60) Provisional application No. 63/337,448, filed on May 2, 2022.

(51) Int. Cl. C07D 241/12; C07D 487/04; C09K 11/06; H10K 50/11; H10K 50/19; H10K 85/60

(52) U.S. Cl. CPC C07D 241/12; C07D 487/04; C09K 11/06; H10K 85/654; H10K 85/6572; C09K 2211/1007; C09K 2211/1018; H10K 50/11; H10K 50/19

(57) ABSTRACT

Provided are luminescent organic compounds containing a benzoyl group covalently bonded to a pyrazine group, along with methods of synthesizing such compounds. The luminescent compounds can be used in organic light-emitting diodes (OLEDs), which can be used in OLED lights or in OLED displays, such as television screens. By varying the voltage applied to such luminescent organic compounds, their emissions spectra can be varied, such as to produce white light or to produce a desired color of light.

### CROSS-REFERENCE TO RELATED APPLICATIONS

This application claims the benefit of priority to U.S. Provisional Application No. 63/337,448, filed May 2, 2022, the disclosure of which is incorporated herein by reference in its entirety.

### GOVERNMENT RIGHTS

This invention was made with government support under grant number 1752821 awarded by the National Science Foundation. The government has certain rights in the invention.

### INTRODUCTION

Luminescent compounds have numerous applications due to their ability to emit light in response to various conditions. For instance, electroluminescent compounds emit light in response to an electrical current being passed through the compound, whereas photoluminescent compounds absorb photons and then emit light, typically of a different wavelength. Electroluminescent compounds can be used in light sources such as light-emitting diodes (LEDs) in televisions, smartphones, and flashlights. Organic light-emitting diodes (OLEDs) are LEDs that use one or more organic compounds as the light-emitting compound.

Electronic visual displays such as televisions, computer monitors, and smartphones can display information by having a range of pixels, usually arranged in a rectangular grid. For a monochromatic display, each pixel can have a single light emitting module. For instance, with the Apple IIe computer of 1983 each pixel was either illuminated as green or non-illuminated as black. In order to show a range of colors, however, each pixel can have multiple sub-pixels. For instance, a pixel could include a three sub-pixels that each emit either red, green, or blue light. By varying the intensity of each sub-pixel, a wide range of colors can be shown. However, multichromatic displays with multiple sub-pixels per pixel can be more expensive and complex than monochromatic displays with only one sub-pixel per pixel.

In addition, different compounds can emit light within either a narrower range of wavelengths or within a broader range. Sometimes the breadth of the emitted wavelengths can be described by the full-width at half-maximum (FWHM) of the emission spectrum. Compounds that emit relatively monochromatic light within a narrow wavelength range can be advantageous in some applications, and in some cases can have a more appealing visual appearance.

### SUMMARY

Provided are luminescent organic compounds containing a benzoyl group covalently bonded to a pyrazine group, along with methods of synthesizing such compounds. The luminescent compounds can be used in organic light-emitting diodes (OLEDs), which can be used in OLED lights or in OLED displays, such as television screens. By varying the voltage applied to such luminescent organic compounds, their emissions spectra can be varied, such as to produce white light or to produce a desired color of light.

### BRIEF DESCRIPTION OF THE DRAWINGS

FIG. 1 shows the emission spectrum of a benzoyl-pyrazine compound with a FWHM of about 5 nm.

FIG. 2 shows the emission spectrum of references DABNA-type compounds with a FWHM of 32 or 33 nm.

FIG. 3A shows a relationship between excitation and emission wavelengths for a benzoyl-pyrazine compound.

FIG. 3B shows some of the emission spectra corresponding to FIG. 3

FIG. 3C shows a graph of the FWHM compared to excitation wavelength of a benzoyl-pyrazine compound.

FIG. 4A shows a photograph of non-excited and photoexcited solutions of a benzoyl-pyrazine compound at high concentration.

FIG. 4B shows a photograph of non-excited and photoexcited solutions of a benzoyl-pyrazine compound at low concentration.

FIG. 5 shows how emission wavelength changes due to changes in concentration.

FIG. 6 shows a proposed OLED with a single light emitting layer that includes a benzoyl-pyrazine compound.

FIG. 7 shows a reference device with three different modules that emit either red, green, or blue light.

FIG. 8 shows a reference device wherein multiple light emitting compounds are combined together in a single light emitting layer.

FIG. 9 shows the 1931 CIE XYZ color space (CIE: International Commission on Illumination or Commission internationale de l′éclairage in French). Moving clockwise from 460 nm to 620 are colors blue, cyan, green, yellow, orange, and red. Moving left to right along the bottom edge transitions from blue to purple to magenta to red. The colors fade towards white near the center of the shape.

FIG. 10 shows a flow diagram of methods of synthesizing benzoyl-pyrazine compounds.

### DETAILED DESCRIPTION

Provided are luminescent organic compounds containing a benzoyl group covalently bonded to a pyrazine group, along with methods of synthesizing such compounds. The luminescent compounds can be used in organic light-emitting diodes (OLEDs), which can be used in OLED lights or in OLED displays, such as television screens. By varying the voltage applied to such luminescent organic compounds, their emissions spectra can be varied, such as to produce white light or to produce a desired color of light.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a droplet” includes a plurality of such droplets and reference to “the discrete entity” includes reference to one or more discrete entities, and so forth. It is further noted that the claims may be drafted to exclude any element, e.g., any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed. To the extent the definition or usage of any term herein conflicts with a definition or usage of a term in an application or reference incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

“Alkyl” refers to monoradical, branched or linear, non-cyclic, saturated hydrocarbon group. Exemplary alkyl groups include methyl, ethyl, n-propyl, isopropyl, n-butyl, isobutyl, t-butyl, octyl, decyl, cyclopentyl, and cyclohexyl. In some cases the alkyl group comprises 1 to 24 carbon atoms, such as 1 to 18 carbon atoms or 1 to 12 carbon atoms. The term “lower alkyl” refers to an alkyl groups with 1 to 6 carbon atoms.

“Alkenyl” refers to a monoradical, branched or linear, non-cyclic hydrocarbonyl group that comprises a carbon-carbon double bond. Exemplary alkenyl groups include ethenyl, n-propenyl, isopropenyl, n-butenyl, isobutenyl, octenyl, decenyl, tetradecenyl, hexadecenyl, eicosenyl, and tetracosenyl. In some cases the alkenyl group comprises 1 to 24 carbon atoms, such as 1 to 18 carbon atoms or 1 to 12 carbon atoms. The term “lower alkenyl” refers to an alkyl groups with 1 to 6 carbon atoms.

“Alkynyl” refers to a monoradical, branched or linear, non-cyclic hydrocarbonyl group that comprises a carbon-carbon triple bond. Exemplary alkynyl groups include ethynyl and n-propynyl. In some cases the alkenyl group comprises 1 to 24 carbon atoms, such as 1 to 18 carbon atoms or 1 to 12 carbon atoms. The term “lower alkenyl” refers to an alkyl groups with 1 to 6 carbon atoms.

“Cycloalkyl” refers to a monoradical, cyclic, saturated hydrocarbon group. Similarly, “cycloalkenyl” refers to a monoradical and cyclic group having carbon-carbon double bond whereas “cycloalkynyl” refers to a monoradical and cyclic group having carbon-carbon triple bond.

“Heterocyclyl” refers to a monoradical, cyclic group that contains a heteroatom (e.g. O, S, N) as a ring atom and that is not aromatic (i.e. distinguishing heterocyclyl groups from heteroaryl groups). Exemplary heterocyclyl groups include piperidinyl, tetrahydrofuranyl, dihydrofuranyl, and thiocanyl.

“Aryl” refers to an aromatic group containing at least one aromatic ring, wherein each of the atoms in the ring are carbon atoms, i.e. none of the ring atoms are heteroatoms (e.g. O, S, N). In some cases the aryl group has a second aromatic ring, e.g. that is fused to the first aromatic ring. Exemplary aryl groups are phenyl, naphthyl, biphenyl, diphenylether, diphenylamine, and benzophenone.

“Heteroaryl” refers to an aromatic group containing at least one aromatic ring, wherein at least one of the atoms in the ring is a heteroatom (e.g. O, S, N). Exemplary heteroaryl groups include furyl, thiophenyl, imidazoyl, and pyrimidinyl.

The term “substituted” refers the removal of one or more hydrogens from an atom (e.g. from a C or N atom) and their replacement with a different group. For instance, a hydrogen atom on a phenyl (—C₆H₅) group can be replaced with a methyl group to form a —C₆H₄CH₃ group. Thus, the —C₆H₄CH₃ group can be considered a substituted aryl group. As another example, two hydrogen atoms from the second carbon of a propyl (—CH₂CH₂CH₃) group can be replaced with an oxygen atom to form a —CH₂C(O)CH₃ group, which can be considered a substituted alkyl group. However, replacement of a hydrogen atom on a propyl (—CH₂CH₂CH₃) group with a methyl group (e.g. giving —CH₂CH(CH₃)CH₃) is not considered a “substitution” as used herein since the starting group and the ending group are both alkyl groups. However, if the propyl group was substituted with a methoxy group, thereby giving a —CH₂CH(OCH₃)CH₃ group, the overall group can no long be considered “alkyl”, and thus is “substituted alkyl”. Thus, in order to be considered a substituent, the replacement group is a different type than the original group. In addition, groups are presumed to be unsubstituted unless described as substituted. For instance, the term “alkyl” and “unsubstituted alkyl” are used interchangeably herein.

In some cases, the substitutions can themselves be further substituted with one or more groups. For example, the group —C₆H₄CH₂CH₃ can be considered as substituted aryl, i.e. an aryl group substituted with the ethyl, which is an alkyl group. Furthermore, the ethyl group can itself be substituted with a pyridyl group to form —C₆H₄CH₂CH₂C₅H₅N, wherein —C₆H₄CH₂CH₂C₅H₅N can also be considered as a substituted aryl group as the term is used herein.

Exemplary substituents include alkyl, alkenyl, alkynyl, cycloalkyl, heterocyclyl, aryl, heteroaryl, acyl, alkoxy, amino, halo, carboxy, sulfonyl, hydroxyl, oxo, thioketo, cyano, nitro, azido, and substituted versions thereof.

Diradical groups are also described herein, i.e. in contrast to the monoradical groups such as alkyl and aryl described above. The term “alkylene” refers to the diradical version of an alkyl group, i.e. an alkylene group is a diradical, branched or linear, cyclic or non-cyclic, saturated hydrocarbon group. Exemplary alkylene groups include diylmethane (—CH₂—, which is also known as a methylene group), 1,2-diylethane (—CH₂CH₂—), and 1,1-diylethane (i.e. a CHCH₃ fragment where the first atom has two single bonds to other two different groups). The term “arylene” refers to the diradical version of an aryl group, e.g. 1,4-diylbenzene refers to a C₆H₄ fragment wherein two hydrogens that are located para to one another are removed and replaced with single bonds to other groups. The terms “alkenylene”, “alkynylene”, “heteroarylene”, and “heterocyclene” are also used herein.

“Acyl” refers to a group of formula —C(O)R wherein R is alkyl, alkenyl, or alkynyl. For example, the acetyl group has formula —C(O)CH₃.

“Alkoxy” refers to a group of formula —O(alkyl). Similar groups can be derived from alkenyl, alkynyl, aryl, heteroaryl, and other groups.

“Amino” refers to the group —NRR′ wherein R and R′ are independently hydrogen or nonhydrogen substituents, with nonhydrogen substituents including, for example, alkyl, alkenyl, alkynyl, aryl, heteroaryl, and substituted versions thereof.

“Halo” and “halogen” refer to the chloro, bromo, fluoro, and iodo groups.

“Carboxyl”, “carboxy”, and “carboxylate” refer to the —CO₂H group and salts thereof.

Unless otherwise specified, reference to an atom is meant to include all isotopes of that atom. For example, reference to His meant to include ¹H, ²H (i.e., D) and ³H (i.e., T), and reference to C is meant to include ¹²C and all isotopes of carbon (e.g. ¹³C). Unless specified otherwise, groups include all stereoisomers.

Pyrazine is a compound with the formula:

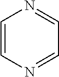

Benzoyl is a group with the formula:

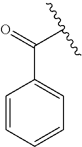 [SMILES: *C(=O)c1ccccc1]

Full width at half maximum (FWHM) refers to the difference in two values of the independent variable when the dependent variable is equal to half its maximum value. In other words, the FWHM is the width of a peak at half its maximum intensity.

### Compounds

Provided are luminescent benzoyl-pyrazine compounds, i.e. containing a benzoyl group covalently bonded to a pyrazine group. Such benzoyl and pyrazine groups are independently substituted or unsubstituted. In some cases, a single benzoyl group is bonded to the pyrazine, and in other cases two benzoyl groups are bonded to the same pyrazine.

In some cases, the compound has formula (Ia), (Ib) or (Ic):

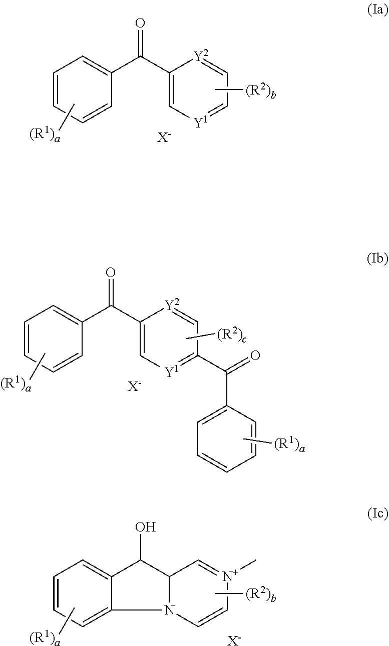

wherein:
X⁻ is a non-coordinating anion;
Y¹ is NCH₃⁺ and Y² is N, or Y¹ is N and Y² is NCH₃⁺;
each a is independently 0, 1, 2, 3, 4, or 5;
b is 0, 1, 2, or 3;
c is 0, 1, or 2; and
each R¹ and R² is independently selected from the group consisting of alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, substituted heterocyclyl, halo, hydroxy, carboxy, alkoxy, substituted alkoxy, amino, and acyl.

In some cases, the compound has formula (Ia). In some cases, the compound has formula (Ib). In some cases, the compound has formula (Ic).

In certain embodiments, Y¹ is NCH₃⁺ and Y² is N, or Y¹ is N and Y² is NCH₃⁺. In other words, one of such Y groups is a nitrogen atom, whereas the other Y group is a nitrogen atom that has been methylated to form the corresponding cationic group. This cationic group corresponds to the anionic group X⁻, which is a non-coordinating anion. Exemplary non-coordination anions include, but are not limited to, BF₄⁻, PF₆⁻, ClO₄⁻, or B(Ar^{F})₄⁻, wherein each Ar^{F} is independently an aryl group substituted with at least one fluorine or at least one trifluoromethyl group.

In some cases, the Y² group, which is ortho to the carbonyl group, is NCH₃⁺. This option is referred to as having the structure of formula (1a-ortho). In some cases, the Y¹ group, which is meta to the carbonyl group, is NCH₃⁺. This option is referred to as having the structure of formula (1a-meta).

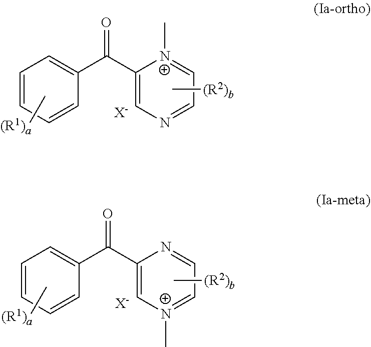

In some cases, b is 0 and therefore the pyrazine group is not substituted with any other group besides the benzoyl group. In other cases, b ranges from 1 to 3, and therefore other groups are attached to the pyrazine. Such “b” groups can be the same or different from each other.

In some cases, the compound has formula (1b), wherein two benzoyl groups are bonded to the pyrazine ring at locations para to one another.

In some instances, “a” ranges from 1 to 5, and therefore the benzoyl group's phenyl ring is substituted with 1 to 5 R¹ groups. Exemplary R¹ groups include, but are not limited to, H, alkyl (e.g. methyl, ethyl, n-propyl, i-propyl), alkoxy (e.g. methoxy, ethoxy), and halo (e.g. F, Cl, Br). In some instances, “a” is 1 and the R¹ group is located para to the carbonyl group.

In some cases, the compound of formula (I) has formula (II):

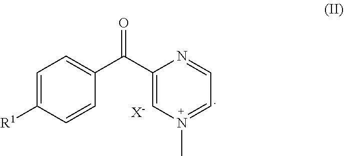

In some embodiments of formula (II), R¹ is selected from H, methyl, ethyl, methoxy, alkoxy, F, Cl, Br, aryl, substituted aryl, heteroaryl, substituted heteroaryl, heterocyclyl, substituted heterocyclyl, and acyl.

In some cases, the compound has formula (Ib), wherein two benzoyl groups are bonded to the pyrazine ring at locations para to one another.

In some instances, the compound has a single benzoyl group bonded to the pyrazine group, but also one of the Y groups is an N-oxide group instead of a N-methyl group and the X⁻ group that was previously described is not present. In such cases, the compound can have formula (VII) wherein Y³ is N⁺—O⁻ and Y⁴ is N, or Y³ is N and Y⁴ is N⁺—O⁻:

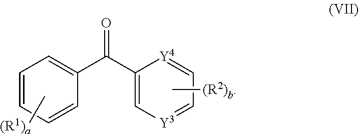

In some embodiments of formula (VII), R¹ is selected from H, methyl, ethyl, methoxy, alkoxy, F, Cl, Br, aryl, substituted aryl, heteroaryl, substituted heteroaryl, heterocyclyl, substituted heterocyclyl, and acyl. In some instances an R¹ group is located para to the carbonyl group. In some cases an R¹ group is located meta or ortho to the carbonyl group.

In some cases, a compound of formula (Ia) (e.g., Ia-meta) or formula (II) can be converted to a compound of formula (Ic) by exposure to light. In some instances, a compound of formula (Ia) (e.g., Ia-meta) or formula (II) can be converted to a compound of formula (Ic) by exposure to light having a wavelength of 280 nm-400 nm.

The luminescent benzoyl-pyrazine compounds can be photoluminescent (e.g. fluorescent, phosphorescent), electroluminescent, or a combination thereof. In such cases, the light emitted during the luminescent emissions can have a particular emission spectrum. The emission properties can be present when the compound is a thin film, is part of a solution, or both. Thin films can be generated by drop-casting solutions of the materials onto a solid surface and by spin-coating a solid surface with a solution. In some cases, the luminescent benzoyl-pyrazine compounds have particular emission spectra in acetonitrile.

In some cases, the compounds have an emission spectrum that is relatively narrow. For instance, the emission spectrum can have a full width at half maximum (FWHM) of 50 nm or less, such as 40 nm or less, 30 nm or less, 20 nm or less, 10 nm or less, 8 nm or less, 6 nm or less, or 5 nm or less. In some cases, the emission maximum has a wavelength ranging from 380 nm to 750 nm, such as from 450 nm to 675 nm or from 500 nm to 650 nm. Such emissions can be fluorescent emissions, i.e. emissions that are produced after absorption of light. The spectrum of light that is absorbed by and excites the benzoyl-pyrazine compound is referred to as the excitation spectrum. In some cases the excitation spectrum has an excitation maximum ranging from 400 to 750 nm, e.g. 400 to 450 nm, 450 to 485 nm, 485 to 500 nm, 500 to 565 nm, 565 to 590 nm, 590 to 625 nm, and 625 to 750 nm.

### Methods of Synthesizing Compounds

Provided are methods of synthesizing the luminescent benzoyl-pyrazine compounds. As described above, in some cases the compounds are within the scope of formula (Ia), (Ib), or (VII). For the single-addition products of formula (Ia), as described above, if the ortho nitrogen is methylated, then the compound has formula (Ia-ortho), whereas if the meta nitrogen is methylated then the compound has formula (Ia-meta). Compounds of formula (Ib) can be referred to as double-addition products because two benzoyl groups are bonded to a single pyrazine group.

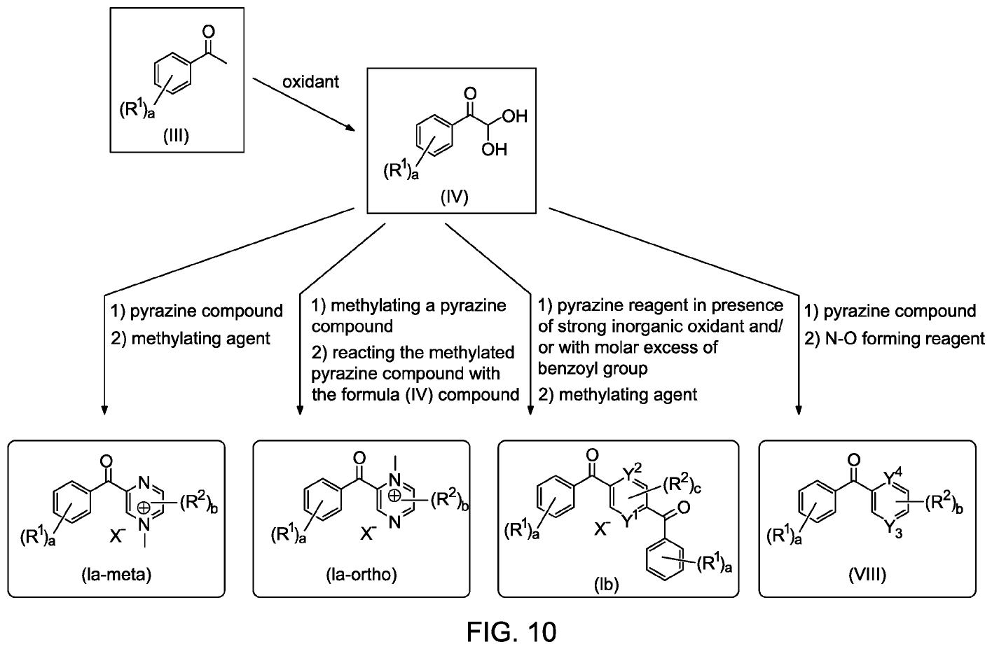

FIG. 10 shows a flow diagram of methods of synthesizing compounds of formulas (Ia-meta), (Ia-ortho), (Ib), and (VIII). First, a compound of formula (III) is reacted with an oxidant to generate a compound of formula (IV), which has a geminal diol group. The conversion of the formula (III) compound to a formula (IV) compound involves the conversion of the —C(O)CH₃ group to a —C(O)C(OH)₂H group. In some cases the oxidant is selenium (IV) oxide. Next, this geminal diol compound of formula (IV) is reacted under different conditions to generate the (Ia-meta), (Ia-ortho), (Ib), or (VIII) compounds.

The method of synthesizing the formula (Ia-meta) compound comprise:
reacting a compound of formula (III) with an oxidant to generate a compound of formula (IV);
reacting the formula (IV) compound with a compound of formula (V) to generate an intermediate compound; and
reacting the intermediate compound with a methylating agent to generate the formula (Ia-meta) compound,
wherein formula (V) is:

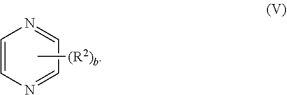

In some cases the intermediate compound is isolated, e.g. and optionally purified, and in other cases the intermediate is reacted with the methylating agent without isolation or purification. For instance, the intermediate compound can have the structure:

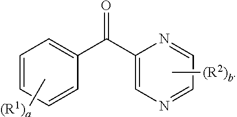

Synthesizing the formula (Ia-ortho) compound comprise:
reacting a compound of formula (III) with an oxidant to generate a compound of formula (IV);
reacting a compound of formula (V) with a methylating agent to generate a compound of formula (V-methylated); and
reacting the compound of formula (V-methylated) with the formula (IV) compound to form the generate (Ia-ortho) compound,
wherein formula (V-methylated) is:

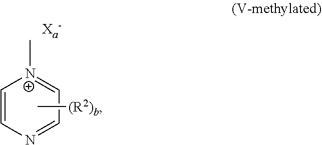

wherein Xₐ⁻ is an anion.

In some cases, Xₐ⁻ is the same anion as the X non-coordinating anion of formula (Ia). In other cases, Xₐ⁻ is a different anion than X, i.e. Xₐ⁻ can be replaced with X in another synthetic step. In some cases, Xₐ⁻ is a non-coordinating anion. Exemplary non-coordination anions include, but are not limited to, BF₄⁻, PF₆⁻, ClO₄⁻, or B(Ar^{F})₄⁻, wherein each Ar^{F} is independently an aryl group substituted with at least one fluorine or at least one trifluoromethyl group

In other words, whereas in the synthesis of the meta compounds the pyrazine group is bonded to the benzoyl group and then methylated, in the synthesis of the ortho compounds the pyrazine is first methylated and then bonded to the benzoyl group.

As used herein, the term “methylating agent” refers to a compound that can react to form a N—CH₃ group on one of the nitrogen atoms of a pyrazine group. When synthesizing either the formula (Ia-meta) or (Ia-ortho) compounds, in some cases the methylating agent comprises a group of formula (VI):

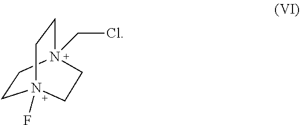

In some cases, the formula (VI) group has an ionic bond to one or more non-coordinating anions, i.e. to balance the two positive charges. For instance, the formula (VI) group can be part of a compound that contains two BF₄⁻ groups. Other exemplary non-coordinating anions include PF₆⁻, ClO₄⁻, or B(Ar^{F})₄⁻.

As described above, methods for synthesizing a formula (Ib) compound are also provided, i.e, wherein formula (Ib) compounds are the result of the addition of two benzoyl groups to a single pyrazine group.

Methods for synthesizing a formula (Ib) compound comprise:
reacting a compound of formula (III) with an oxidant to generate a compound of formula (IV);
reacting the formula (IV) compound with a compound of formula (V) to generate an intermediate compound; and
reacting the intermediate compound with a methylating agent to generate the formula (Ib) compound,
wherein one or more of: (i) the molar ratio of the compound of formula (IV) to the compound of formula (V) is greater than 1.1, and (ii) reacting the compound of formula (IV) with the compound of formula (V) is performed with an inorganic oxidant.

For instance, when the molar ratio of the compound of formula (IV) to the compound of formula (V) is greater than 1.1, the double addition product of formula (Ib) can be favored over the single-addition product of formula (Ia-meta). In some cases the molar ratio can range from 1.1 to 10, such as from 1.5 to 5 or 2 to 4.

The use of an inorganic oxidant, such as a strong inorganic oxidant such as an inorganic persulfate, can also favor the double addition product to generate the formula (Ib) compound instead of the formula (Ia-meta) compound. In some cases the inorganic persulfate is sodium persulfate, potassium persulfate, or ammonium persulfate.

Methods for synthesizing a compound of formula (VII) comprise:
reacting a compound of formula (III) with an oxidant to generate a compound of formula (IV);
reacting the compound of formula (IV) with a compound of formula (V) and an N-oxide forming reagent to generate the formula (VII) compound.

In some cases, the formula (IV) compound is first reacted with the formula (V) compound to generate an intermediate, and then the intermediate is reacted with the N-oxide forming reagent to generate the formula (VII) compound. In some cases this reaction order generates an N-oxide group at the meta nitrogen, e.g. in an analogous manner to the formula of the methylated meta nitrogen of formula (Ia-meta).

As used herein, the term “N-oxide forming reagent” refers to a compound that can react to form an N—O group on one of the nitrogen atoms of a pyrazine group.

In some cases, the formula (V) compound is first reacted with the N-oxide generating reagent to generate an N-oxide containing pyrazine intermediate, and this intermediate is then reacted with the formula (IV) compound to form the formula (VII) product.

In some cases, the formula (IV) compound, the N-oxide forming reagent, and the formula (V) compound are reacted with each other at the same time.

In some cases, the N-oxide forming reagent is meta-chloroperbenzoic acid (mCPBA).

In formulas having a pyrazine with (R²)_{b}, b can be 0, 1, 2, or 3, since there are up to three positions which can be substituted while still allowing the pyrazine group to bond to a single benzoyl group. Other formulas show the (R²)_{c} group, wherein c is 0, 1, or 2 since there are two positions used to bond to the two benzoyl groups.

### Devices and Methods of Use

### OLEDs

Provided are organic light emitting diodes (OLEDs) that include a cathode, an anode, and a light emitting layer located between the cathode and the anode that comprises a luminescent benzoyl-pyrazine compound as described above. An exemplary anode is indium tin oxide (ITO) and an exemplary cathode is a gold-platinum compound (Au—Pt).

The anode can be configured to be relatively transparent in order to minimize the amount of generated light that is absorbed by the anode, wherein ITO absorbs a small fraction of visible light. Such OLEDs can be referred to as bottom emitting OLEDs. In some cases, the cathode can be configured to be relatively transparent, and such OLEDs can be referred to as top emitting OLEDs. Whereas one electrode can be transparent, the other electrode can be configured to be highly reflective to increase the amount of light output.

The OLED can also include an electron transport layer (e.g. PEDOT:PSS), hole transport layer (e.g. PTAA), or a combination thereof. The electron transport layer can be positioned between the cathode and the light emitting layer, whereas the hole transport layer can be positioned between the anode and the light emitting layer. Such transport layers can increase the efficiency, i.e. the amount of light emitted for a certain amount of electricity.

In some instances, the OLED comprises a single light emitting layer. In other words, the OLED has one light emitting layer, but it does not have two or more light emitting layers.

In some cases, the light emitting layer has a single light emitting compound. In other words, the light emitting layer has one light emitting compound, but it does not have two or more light emitting compounds. Stated in another manner, some light emitting layers might have multiple compounds with different chemical structures, whereas single-compound light emitting layers have many different molecules that are each light emitting, but all of such light emitting molecules have the same chemical structure. Since manufacturing does not always generate perfectly pure compositions, “all” compounds having the same chemical structure means that 95% or more of the compounds have the same structure, such as 98% or more.

### OLED Displays

Provided are OLED displays that include an OLED with a light emitting layer that includes a benzoyl-pyrazine compound as described above. Exemplary OLED displays include television screens, smart phone screens, and computer monitors.

For instance, the OLED display can include a housing a first pixel comprising a sub-pixel, wherein the sub-pixel comprises an OLED as described above. In some cases the OLED display includes multiple of such pixels that each comprise a sub-pixel comprising an OLED. For instance, the pixels can be arranged in a grid of rows and columns, such as with a computer monitor. In some cases the OLED display has at least 1,000 pixels, such as at least 10,000 or at least 100,000.

When controlled by computer software, each pixel can define a location within the display, whereas the one or more sub-pixels can be used to display a particular color at the location.

In some cases, the emission spectrum of the benzoyl-pyrazine compound in the light emitting layer will change depending upon the voltage delivered to light emitting layer. The voltage can be changed by an element of the display device, and therefore the OLED receives a changed voltage. In other cases, the voltage delivered to the OLED is constant, but the OLED itself changes the voltage delivered to the light emitting layer. As such, by changing the voltage delivered, the emission spectrum and therefore the color observable by a human can be changed in a controlled manner.

In some cases, a particular voltage is chosen such that the benzoyl-pyrazine compounds in the light emitting layer generate a particular emission spectrum that is interpreted by a human observer as the desired color. In some cases, the delivered voltage is constant during the period of time. In some instances, the constant voltage has a coefficient of variation of 10% or less, such as 5% or less or 1% or less. In some instances, the constant voltage has a maximum voltage that is 110% or less of the minimum voltage, such as 105% or less.

Provided is a method of displaying information with an OLED display, the method comprising delivering voltage to the light emitting layer of the OLED for a period of time. This delivering causes the light emitting layer to emit light for the period of time. Exemplary periods of time include 0.001 second to 0.1 seconds. In some cases, computer monitors and other displays are configured to refresh and change their colors at frequencies of 30 Hz, 60 Hz, or 120 Hz, which correspond to periods of time of about 0.0333 seconds, 0.0166 seconds, and 0.0083 seconds. Thus, in some cases the period of time ranges from 0.008 seconds to 0.035 seconds.

In some cases, a single voltage was found to produce an emission spectrum that corresponds to the desired color. Hence, during the time period, the delivered voltage can be held constant.

In other cases, the desired color cannot be produced by a constant emission spectrum and a constant voltage, and therefore the voltage is varied during the time period in order to produce an average emission spectrum that does correspond to the desired color. Since humans cannot instantaneously detect changes in color (e.g. Potter et al, Attention, Perception, & Psychophysics, 2014; Bodelon et al, Vision Sciences Society Annual Meeting Abstract, 2005), rapid changes in the emission spectrum can be averaged by the eyes and brain of the human so that the desired color is observed.

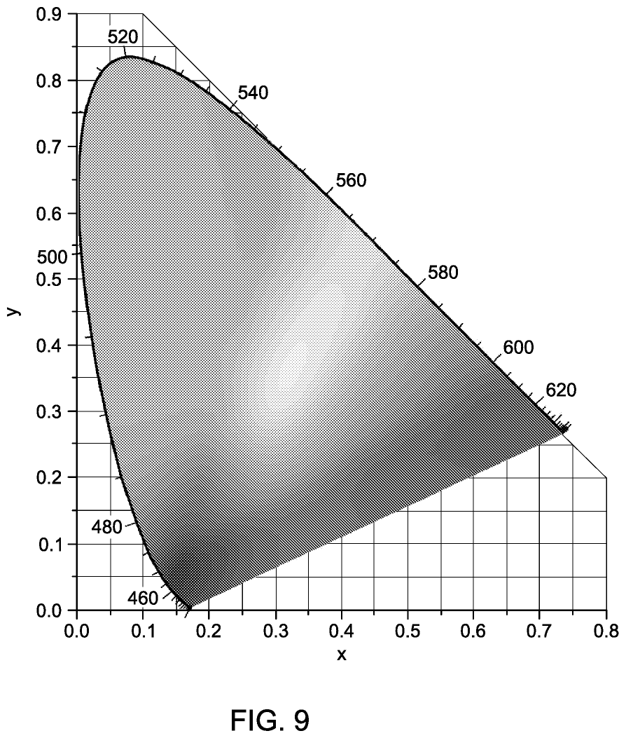

To illustrate how some observable colors cannot necessarily be produced by a single emission spectrum from the benzoyl-pyrazine compounds, the International Commission on Illumination (CIE) 1931 XYZ color space can be considered. FIG. 9 shows the 1931 CIE XYZ color space. The numbers circulating clockwise around the perimeter of the shape refer to the wavelength of monochromatic light that would produce the color at the perimeter of the shape. Beginning at 460 nm, the colors are blue, cyan, green, yellow, orange, and red. Such “spectral colors” are the colors that can be produced by monochromatic light of a single wavelength.

However, colors become less saturated as they move from the perimeter to the interior. In other words, colors fade towards “white” as they move towards the white point at about (0.3, 0.35). In some cases the white light is near CIE standard illuminant D65 in the 1931 CIE XYZ color space, such as within a radius of 0.4, 0.35, 0.3, 0.25, 0.2, 0.15, 0.1, or 0.05. In some cases the white light is within the polygon defined by (0.3, 0.45), (0.375, 0.4), (0.3, 0.3), and (0.26, 0.3). Therefore, the present OLED devices can be configured to produce white light from a single luminescent benzoyl-pyrazine compound.

As such, changing the voltage and thereby the emission spectrum can be used to generate different emission spectra. If cycled quickly enough, such spectra will be combined by the eyes and mind of a human observer to produce a desired color, such as white or approximately white. In some instances, the average of the light emitted over the period of time is white light. To average light, the intensity of light at a particular wavelength is recorded at multiple time periods, and the intensities are averaged to give the average intensity at the particular wavelength. This procedure is repeated for each measured wavelength, and the averages are combined to form the overall average emission spectrum.

As such, the method can include varying the delivered voltage during the time period. For instance, the voltage can be changed between two or more distinct voltages, or the voltage can be continuously varied (i.e. a voltage sweep) from a first voltage, through intermediate voltages, and to a final voltage. For instance, Zaumseil published a review that describes methods of varying the voltage (e.g. voltage sweep) that can be used with the OLED displays described herein (Zaumseil, “Recent Developments and Novel Applications of Thin Film, Light-Emitting Transistors”, Advanced Functional Materials, 2019, 30, 20, 1905269, doi:10.1002/adfm.201905269). Zaumseil is incorporated herein by reference.

As such, the color observed by a human can be the average of the emission spectra from the different voltages. The voltage can also be cycled two or more times during the time period, i.e. the pattern of voltage change can be repeated a second time, a third time, a fourth time, or other additional times. In some cases, the voltage is cycled 5 or more times during the period of time, such as 10 or more, 20 or more, or 50 or more.

In some cases, the emission maximum is changed by 50 nm or more during the period of time, such as by 100 nm or more or 150 nm or more.

In some cases, the first pixel comprises a single sub-pixel. As such, a certain color (e.g. white) can be produced without the need for multiple sub-pixels per pixel that have different emission spectra.

In some cases, the sub-pixel has a single light emitting layer. Thus, a certain color (e.g. white) can be produced without the need to stack multiple light emitting layers that each have different emission spectra.

In some cases, the light emitting layer has a single light emitting compound. Hence, a certain color (e.g. white) can be produced without the need for multiple light emitting compounds with different emission spectra to be located within the same light emitting layer.

### OLED Lights

Provided are OLED lights that include a housing an OLED as described above. Examples of such OLED lights include a handheld flashlight and a lightbulb configured for installation in a lamp or in the ceiling of a room.

The OLED light can be configured to quickly vary the voltage delivered to the OLED and thereby vary the emission spectrum of the OLED, e.g. as described to generate white light for OLED displays.

The OLED light can be configured such that a user can select a desired color. For instance, the user can select a desired color on a smartphone application, and the smartphone can electronically instruct the OLED light through wireless communication to choose a constant or varying voltage to produce the desired color.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

### Example 1: Synthesis of Compounds

Compounds were synthesized according to scheme below. Compounds wherein R was H, Me, and F were synthesized. In particular, the acetophenone derivative was reacted with selenium (IV) oxide to oxidize the methyl group to a geminal diol group. Next, the diol was reacted with pyrazine in the presence of 1-Chloromethyl-4-fluoro-1,4-diazoniabicyclo[2.2.2]octane bis(tetrafluoroborate) to give the BF₄⁻ salt of the corresponding product.

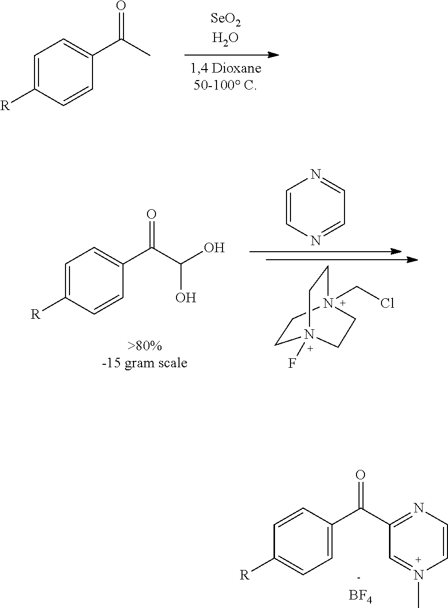

It was also observed that in some cases the regioselectivity of methylation was different from the compound shown above. In other words, the methyl group was attached to the top nitrogen rather than the bottom nitrogen. It was observed that if benzoylation is done first, the subsequent methylation occurs at the nitrogen farthest away from the benzoyl group. In contrast, it was observed that if methylation is performed first, the benzoyl group is then added to the carbon directly adjacent to the methylated nitrogen. Example 2: Photoluminescent properties of thin films

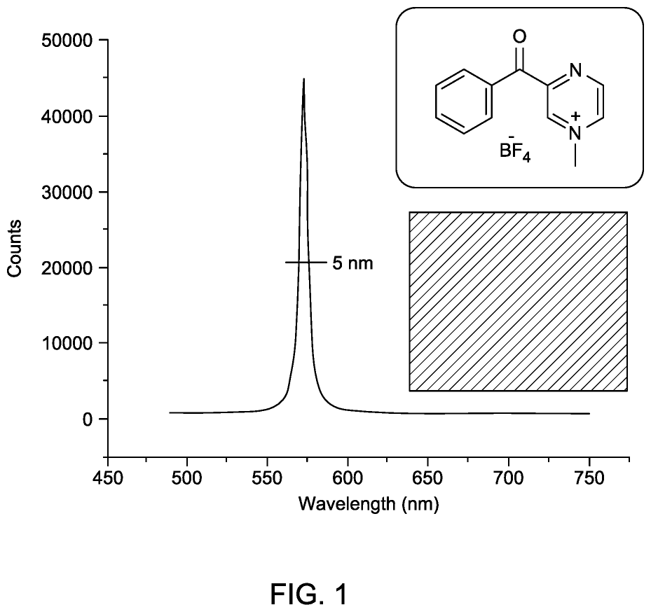

Thin films of the compounds of Example 1 were deposited onto a substrate and subjected to photoexcitation. As shown in FIG. 1 the compound with R═H showed an emission spectrum centered at about 575 nm. Notably, the FWHM of the emission was about 5 nm. Compounds with other R groups were found to have similarly narrow FWHM values.

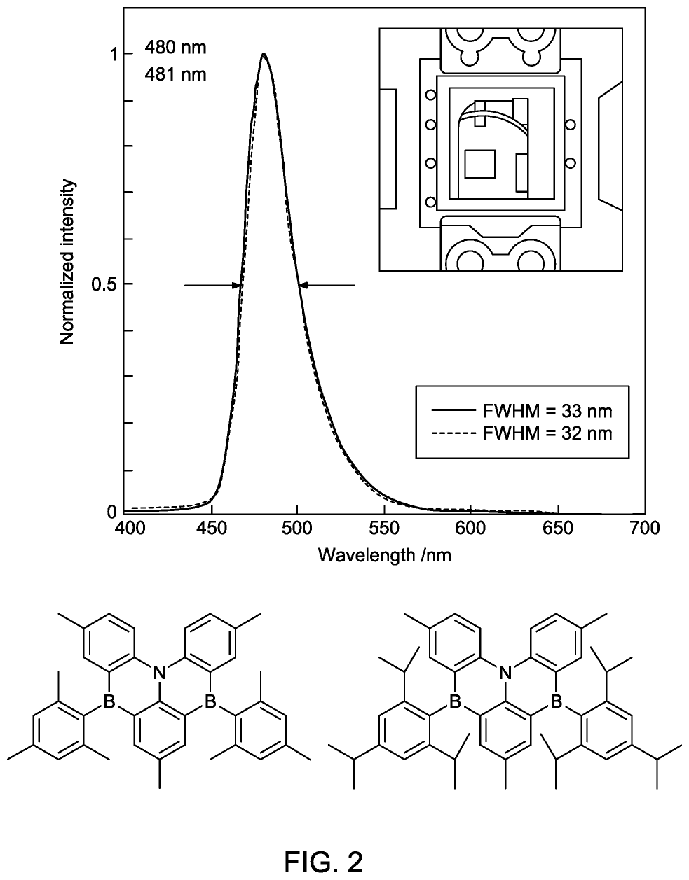

For reference, compounds with relatively narrow emission spectra were also tested in order to allow comparison with the compounds of Example 1. Two compounds, which are derivatives of DABNA1, were selected based on the review article by Ha et al (NPG Asia Materials, 2021, 13, 53, doi:10.1038/s41427-021-00318-8). The emission spectrum of these DABNA compounds was recorded and are shown in FIG. 2, and had FWHM of 32 nm or 33 nm.

As such, compared to known LEDs with spectra widths of about 30 nm at the narrowest, (Ha et al; Zhang et al, Nature Communications, 2020, 11, 2826, doi:10.1038/s41467-020-16659-x), the present Example 1 compounds have narrower FWHM emission spectra.

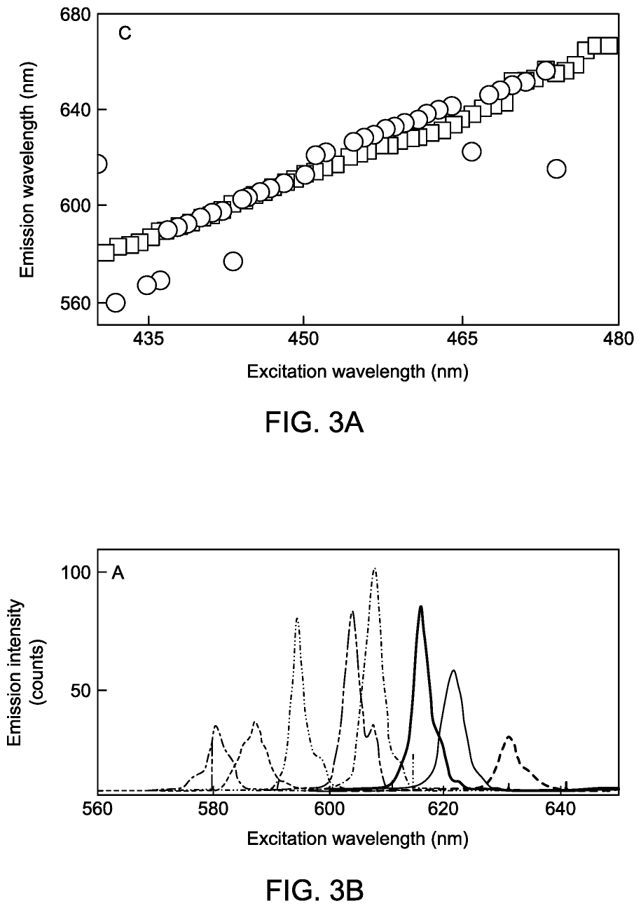

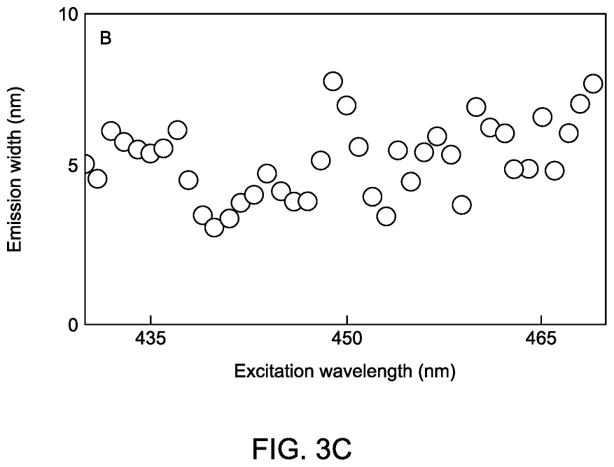

In addition, the effect of different excitation wavelengths on emission wavelength and peak width was also studied. As shown in FIG. 3A, excitation wavelengths of about 420 nm to about 475 nm gave emission maximum wavelengths of about 560 nm to about 650 nm, wherein most data points fell along a relatively straight line. Some of the data points from FIG. 3A were used to create FIG. 3B, which shows eight exemplary emission spectra. The emission spectra were also analyzed for the FWHM peak width, and the results are shown in FIG. 3C. In particular, excitation wavelengths of about 425 nm to about 470 nm gave FWHM values ranging from about 3 nm to about 8 nm.

### Example 3: Photoluminescent Properties of Dissolved Compounds

The solution photoluminescent measurement was done using Princeton Instruments SP2300i spectrometer. The solution was excited at wavelengths in the range of 430-700 nm using an NKT Photonics Super-K laser. The emission from the sample was detected by a thermoelectrically cooled deep depletion and low noise charge coupled detector coupled to the spectrometer and viewed using the LightField software, and the data was plotted and analysed in Origin lab.

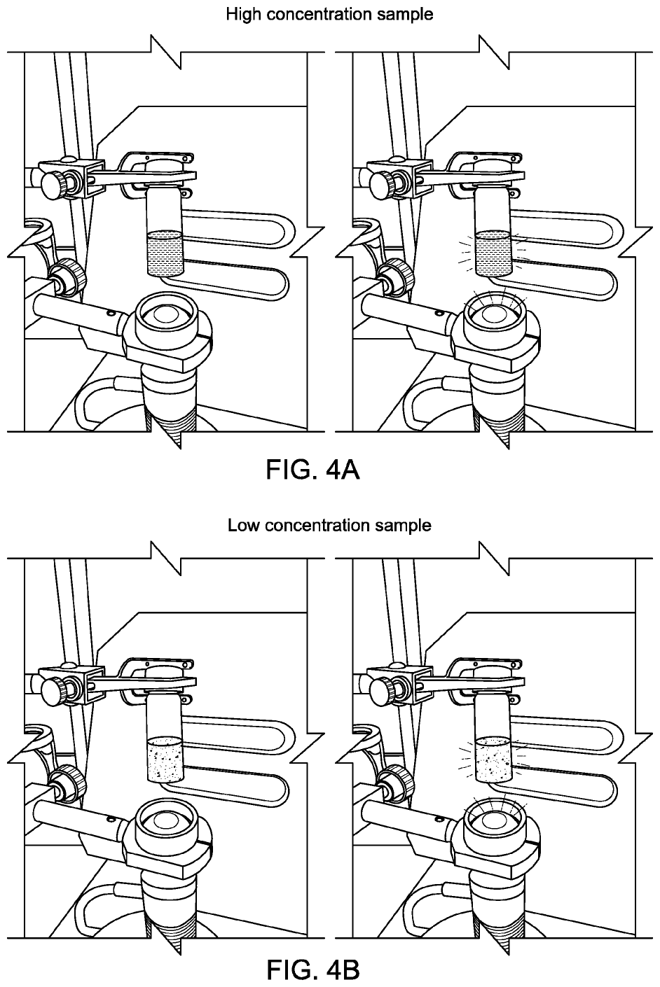

The compounds of Example 1 were dissolved acetonitrile and excited by light with a wavelength of 354 nm. FIG. 4A shows a solution of the R═F compound in normal room light (left) and the same solution when excited by the 354 nm light source (right). FIG. 4B shows a solution of the same R═F compound, but at a lower concentration, with the non-excited solution on the left and the excited solution on the right. As shown in FIG. 1B, the solution was light pink in color when non-excited, but cyan or blue when excited.

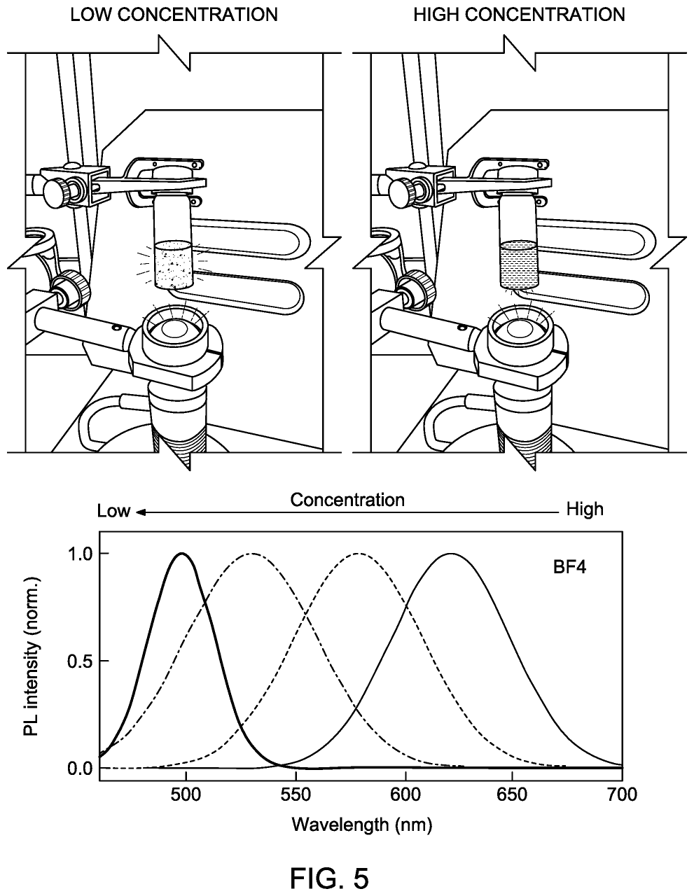

FIG. 5 shows the two images of the excited R═F solutions from FIGS. 4A and 4B adjacent to each other. As shown in the graph of FIG. 5, additional experiments showed that the emission spectrum shifted from a lower wavelength to a higher wavelength when the concentration of the R═F compound was increased, even though the excitation wavelength of 354 nm remained constant.

### Example 4: OLEDs

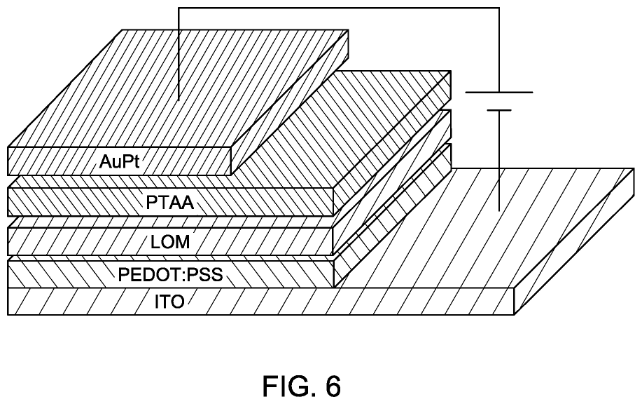

FIG. 6 shows the structure of a possible OLED that includes the compounds of Example 1. In particular, the LOM layer can include the light-emitting compounds of Example 1, whereas the AuPt (gold-platinum) and the ITO (indium tin oxide) are the electrodes. The electron and hole transporting layers of PEDOT:PSS and PTAA are also shown, although these layers can be varied depending upon the energy gap of the compounds being electrically excited.

By varying the voltage applied to the Example 1 compounds in the LOM layer, the emission wavelength could be varied. For instance, changing the energy of the excitation photons (e.g. changing the excitation wavelength) in Example 2 was shown to vary the emission spectrum. As such, varying the electrical voltage could also change the emission spectrum, and therefore the color that the human eye would detect from the OLED. In fact, the voltages could be rapidly cycled so that a variety of colors are shown for certain periods of time, such that average color and therefore the color observed by the human eye could be white, or another non-spectral color.

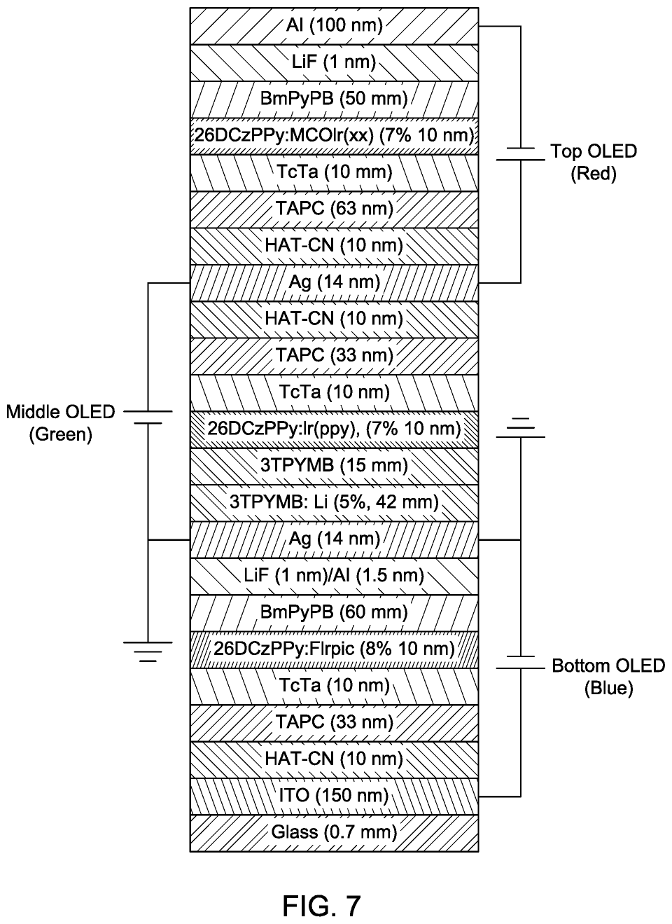

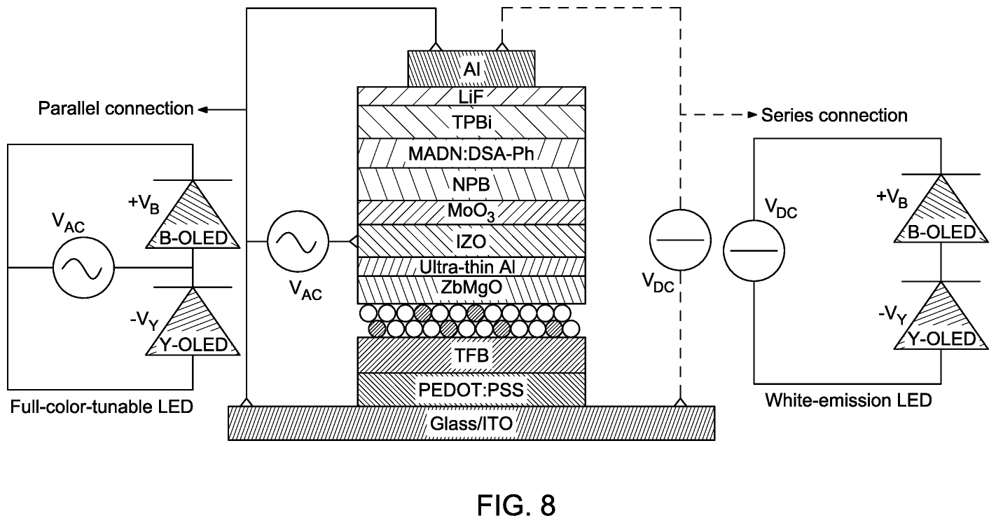

For comparison, FIGS. 7 and 8 shows examples of a traditional OLED devices. In FIG. 7, three different light-emitting modules are arranged vertically such that the top is a red-emitter, the middle is a green-emitter, and the bottom is a blue-emitter (see Lee et al, Op. Exp., 2018, 26, 18351, doi:10.1364/OE.26.018351). By varying the amount of light emitted from each module, different colors can be created. FIG. 8 shows a device wherein the materials emitting at different colors are mixed into a single active layer. However, such devices are more complex than the proposed OLED with a single light emitting layer containing a single light-emitting compound, e.g. a compound of Example 1. Thus, the proposed OLEDs could also be less expensive to manufacture.

### Example 5: Additional Syntheses of Compounds

Additional benzoyl-pyrazine compounds were generated by the coupling of two benzoyl moieties to a single pyrazine group, i.e. in contrast to other methods wherein a single benzoyl group was coupled to the pyrazine. In this double-addition products, it was observed that the benzoyl groups attached para to one another on the pyrazine ring. In some cases, this double addition product was formed when strong inorganic oxidants (e.g. inorganic persulfates) were used.

Separate experiments were performed that generated products wherein one benzoyl and one pyrazine group were coupled, but wherein the resulting compound had an N—O group on the pyrazine ring, i.e. instead of an N—CH₃ group. Meta-chloroperbenzoic acid (mCPBA) was used in slight excess (1.5 equivalents) in dichloromethane at room temperature. Components are mixed in a 0.1 M concentration, collected by filtration or removal of solvent and may be purified via sublimation.

### Example 6: Cyclized Compounds of Formula (Ic)

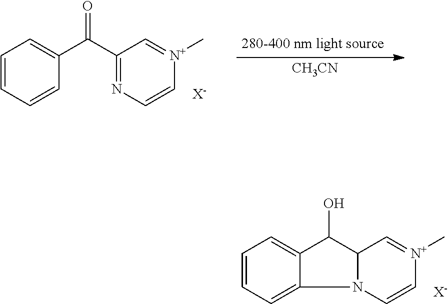

General Procedure for Synthesis of methylpyrazino[1,2]indolium tricycles from benzoyl methylpyrazinium precursors. To a 4 mL borosilicate scintillation vial was added the requisite benzoyl methylpyrazinium starting material. Acetonitrile (2.00 mL) was then added, and the reaction was subjected to constant irradiation with an LED light source between 280-400 nm. Cyclization was monitored periodically by NMR until sufficient conversion was desired. For 280 nm irradiation, complete conversion to the cyclized product was generally observed within 60 minutes; longer wavelengths required longer periods of irradiation to promote full conversion.

In cases of full conversion to the cyclized product, the product is isolated in high purity simply by removing solvent under reduced pressure. In cases of lower conversion, the desired cyclized product is separated from the uncyclized starting materials via silica gel chromatography.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A compound of formula (Ia), (Ib) or (Ic): 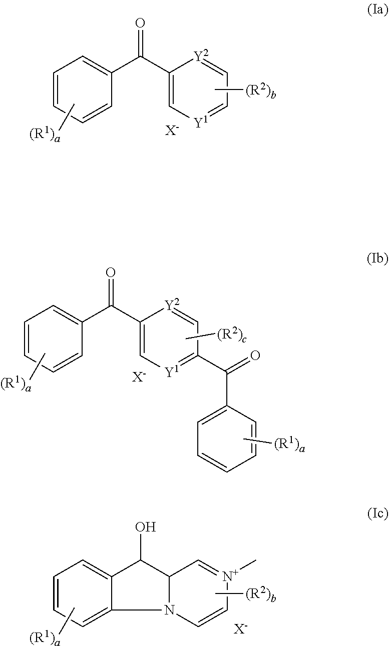
wherein:
X⁻ is a non-coordinating anion;
Y¹ is NCH₃⁺ and Y² is N, or Y¹ is N and Y² is NCH₃⁺;
each a is independently 0, 1, 2, 3, 4, or 5;
b is 0, 1, 2, or 3;
c is 0, 1, or 2; and
each R¹ and R² is independently selected from the group consisting of alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, substituted heterocyclyl, halo, hydroxy, carboxy, alkoxy, substituted alkoxy, amino, and acyl.

2. (canceled)

3. (canceled)

4. (canceled)

5. The compound of claim 1, wherein b is 0.

6. (canceled)

7. (canceled)

8. The compound of claim 1, wherein the compound has formula (II):

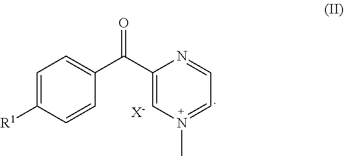

9. The compound of claim 8, wherein R¹ is H, alkyl, alkoxy, halo, aryl, substituted aryl, heteroaryl, substituted heteroaryl, heterocyclyl, substituted heterocyclyl, and acyl

10. The compound of claim 8, wherein X⁻ is BF₄⁻, PF₆⁻, ClO₄⁻, or B(Ar^{F})₄⁻, wherein each Ar^{F} is independently an aryl group substituted with at least one fluorine or at least one trifluoromethyl group.

11. The compound of claim 1, wherein a solution or thin film comprising the compound has a fluorescent emission spectrum with a full width at half maximum (FWHM) of 30 nm or less.

12. (canceled)

13. (canceled)

14. The compound of claim 11, wherein the emission maximum of the fluorescent emission spectrum ranges from 450 nm to 750 nm.

15. The compound of claim 11, wherein the fluorescent emission spectrum is produced after absorption of light having a maximum intensity ranging from 400 nm to 700 nm.

16. (canceled)

17. A method of synthesizing a compound of formula (Ia) or (Ib): 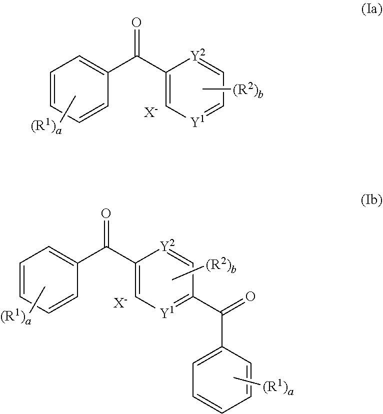
wherein:
X⁻ is a non-coordinating anion;
Y¹ is NCH₃⁺ and Y² is N, or Y¹ is N and Y² is NCH₃⁺;
each a is 0, 1, 2, 3, 4, or 5;
b is 0, 1, 2, or 3;
c is 0, 1, or 2; and
each R¹ and R² is independently selected from the group consisting of alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, substituted heterocyclyl, halo, hydroxy, carboxy, alkoxy, substituted alkoxy, amino, and acyl,
the method comprising:
reacting a compound of formula (III) with an oxidant to generate a compound of formula (IV); and
reacting the compound of formula (IV) to generate the formula (Ia) or (Ib) compound.
wherein formulas (III) and (IV) are:

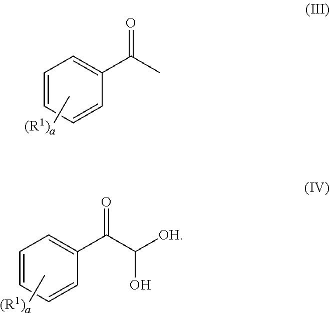

18. The method of claim 17, wherein the method is a method of generating a compound of formula (Ia), wherein the compound of formula (Ia) has the structure of formula (Ia-meta), wherein the method comprises:
reacting the compound of formula (III) with an oxidant to generate a compound of formula (IV);
reacting the compound of formula (IV) with a compound of formula (V) to generate an intermediate compound; and
reacting the intermediate compound with a methylating agent to generate the compound of formula (Ia-meta),
wherein formulas (Ia-meta) and (V) are:

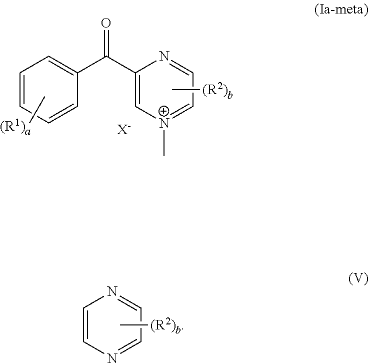

19. The method of claim 17, wherein the method is a method of generating a compound of formula (Ia), wherein the compound of formula (Ia) has the structure of formula (Ia-meta), wherein the method comprises:
reacting the compound of formula (III) with an oxidant to generate the compound of formula (IV);
reacting a compound of formula (V) with a methylating agent to generate a compound of formula (V-methylated); and
reacting the compound of formula (V-methylated) with the compound of formula (IV) to generate the (Ia-ortho) compound,
wherein formulas (Ia-ortho) and (V-methylated) are: 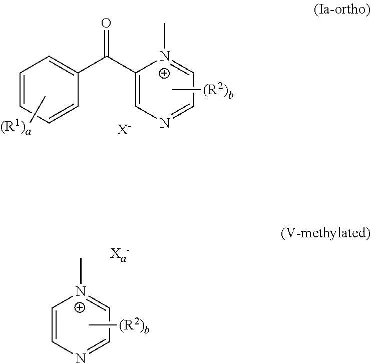
wherein Xₐ⁻ is an anion.

20. The method of claim 17, wherein the method is a method of generating a compound of formula (Ib), wherein the method comprises:
reacting the compound of formula (III) with an oxidant to generate the compound of formula (IV);
reacting the compound of formula (IV) with a compound of formula (V) to generate an intermediate compound; and
reacting the intermediate compound with a methylating agent to generate the compound of formula (Ib),
wherein one or more of: (i) the molar ratio of the compound of formula (IV) to the compound of formula (V) is greater than 1.1, and (ii) reacting the compound of formula (IV) with the compound of formula (V) is performed with an inorganic oxidant,
wherein formula (V) is:

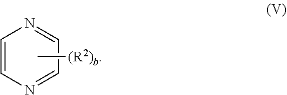

21. The method of claim 17, wherein the methylating agent comprises a group of formula (VI):

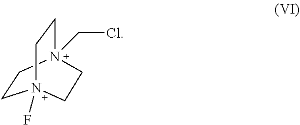

22. (canceled)

23. An organic light-emitting diode (OLED), comprising:
a cathode;
an anode; and
a light emitting layer located between the cathode and the anode and comprising a compound of claim 1.

24. The OLED of claim 23, wherein the OLED comprises a single light emitting layer.

25. The OLED of claim 23, wherein the light emitting layer includes a single light emitting compound.

26. (canceled)

27. (canceled)

28. An organic light-emitting diode (OLED) display, comprising:
a housing; and
a first pixel comprising a sub-pixel, wherein the sub-pixel comprises an OLED according to claim 23.

29. (canceled)

30. (canceled)

31. The OLED display of claim 28, wherein the OLED display is configured to vary the voltage delivered to the light emitting layer of the OLED over a period of time ranging from 0.008 seconds to 0.035 seconds.

32. (canceled)

33. The OLED display of claim 31, wherein varying the delivered voltage comprises cycling the voltage two or more times during the period of time.

34. The OLED display of claim 31, wherein varying the delivered voltage causes the average of the light emitted over a period of time to be white light.

35. The OLED display of claim 34, wherein the white light has an (x, y) value within the polygon defined by (0.3, 0.45), (0.375, 0.4), (0.3, 0.3), and (0.26, 0.3) according to the International Commission on Illumination (CIE) 1931 XYZ color space.

36. The OLED display of claim 34, wherein the white light has an (x, y) value within a radius of 0.25 from CIE standard illuminant D65 according to the CIE 1931 XYZ color space.

37. The OLED display of claim 28, wherein the first pixel comprises a second sub-pixel with a second light-emitting layer that is different than the light-emitting layer.

38. The OLED display of claim 37, wherein the emission maximum of the light-emitting layer and the second light-emitting layer differ by 100 nm or more.

39. (canceled)

40. A method of displaying information with an organic light-emitting diode (OLED) display of claim 28, the method comprising:
delivering a voltage to the light emitting layer of the OLED for a period of time, thereby causing the light emitting layer to emit light for the period of time.

41-49. (canceled)
